(19) United States
(12) Patent Application Publication
(10) Pub. No.: US 2009/0163525 A1
(43) Pub. Date: Jun. 25, 2009

(54) SUBSTITUTED QUINAZOLINES WITH ANTI-CANCER ACTIVITY

(75) Inventors: Brian A. Aquila, Waltham, MA (US); Paul Lyne, Waltham, MA (US); Timothy Pontz, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/295,820

(22) PCT Filed: Apr. 4, 2007
(86) PCT No.: PCT/GB2007/001232

§ 371 Date: Oct. 2, 2008

Related U.S. Application Data
(60) Provisional application No. 60/744,319, filed on Apr. 5, 2006.

(51) Int. Cl. A61K 31/517; C07D 239/80; A61P 35/00

(52) U.S. Cl. 514/266.3; 544/287

(57) ABSTRACT

The invention relates to chemical compounds of the formula (I): or pharmaceutically acceptable salts thereof, which possess B Raf inhibitory activity and are accordingly useful for their anti cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm blooded animal such as man.

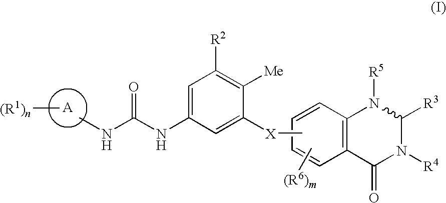

The invention relates to chemical compounds, or pharmaceutically acceptable salts thereof, which possess B-Raf inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm-blooded animal such as man.

The classical Ras, Raf, MAP protein kinase/extracellular signal-regulated kinase kinase (MEK), extracellular signal-regulated kinase (ERK) pathway plays a central role in the regulation of a variety of cellular functions dependent upon cellular context, including cellular proliferation, differentiation, survival, immortalization and angiogenesis (reviewed in Peyssonnaux and Eychene, Biology of the Cell, 2001, 93,3-62). In this pathway, Raf family members are recruited to the plasma membrane upon binding to guanosine triphosphate (GTP) loaded Ras resulting in the phosphorylation and activation of Raf proteins. Activated Rafs then phosphorylate and activate MEKs, which in turn phosphorylate and activate ERKs. Upon activation, ERKs translocate from the cytoplasm to the nucleus resulting in the phosphorylation and regulation of activity of transcription factors such as Elk-1 and Myc.

The Ras/Raf/MEK/ERK pathway has been reported to contribute to the tumorigenic phenotype by inducing immortalisation, growth factor-independent growth, insensitivity to growth-inhibitory signals, ability to invade and metastasis, stimulating angiogenesis and inhibition of apoptosis (reviewed in Kolch et al., Exp.Rev. Mol. Med., 2002, 25 April, http://www.expertreviews.org/02004386h.htm). In fact, ERK phosphorylation is enhanced in approximately 30% of all human tumours (Hoshino et al., Oncogene, 1999, 18, 813-822). This may be a result of overexpression and/or mutation of key members of the pathway.

Three Raf serine/threonine protein kinase isoforms have been reported Raf-1/c-Raf, B-Raf and A-Raf (reviewed in Mercer and Pritchard, Biochim. Biophys. Acta, 2003, 1653, 25-40), the genes for which are thought to have arisen from gene duplication. All three Raf genes are expressed in most tissues with high-level expression of B-Raf in neuronal tissue and A-Raf in urogenital tissue. The highly homologous Raf family members have overlapping but distinct biochemical activities and biological functions (Hagemann and Rapp, Expt. Cell Res. 1999, 253, 34-46). Expression of all three Raf genes is required for normal murine development however both c-Raf and B-Raf are required to complete gestation. B-Raf -/-mice die at E12.5 due to vascular haemorrhaging caused by increased apoptosis of endothelial cells (Wojnowski et al., Nature Genet., 1997, 16, 293-297). B-Raf is reportedly the major isoform involved in cell proliferation and the primary target of oncogenic Ras. Activating somatic missense mutations have been identified exclusively for B-Raf, occurring with a frequency of 66% in malignant cutaneous melanomas (Davies et al., Nature, 2002, 417, 949-954) and also present in a wide range of human cancers, including but not limited to papillary thyroid tumours (Cohen et al., J. Natl. Cancer Inst., 2003, 95, 625-627), cholangiocarcinomas (Tannapfel et al., Gut, 2003, 52, 706-712), colon and ovarian cancers (Davies et al., Nature, 2002, 417, 949-954). The most frequent mutation in B-Raf (80%) is a glutamic acid for valine substitution at position 600. These mutations increase the basal kinase activity of B-Raf and are thought to uncouple Raf/MEK/ERK signalling from upstream proliferation drives including Ras and growth factor receptor activation resulting in constitutive activation of ERK. Mutated B-Raf proteins are transforming in NIH3T3 cells (Davies et al., Nature, 2002, 417, 949-954) and melanocytes (Wellbrock et al., Cancer Res., 2004, 64, 2338-2342) and have also been shown to be essential for melanoma cell viability and transformation (Hingorani et al., Cancer Res., 2003, 63, 5198-5202). As a key driver of the Raf/MEK/ERK signalling cascade, B-Raf represents a likely point of intervention in tumours dependent on this pathway.

AstraZeneca has filed certain international applications directed towards BRaf inhibitors: WO 2005/123696, WO 2006/003378, WO 2006/024834, WO 2006/024836, WO 2006/040568, WO 2006 / 067446 and WO 2006/079791. The present application is based on a class of compound which are novel BRaf inhibitors and it is expected that these compounds could possess beneficial efficacious, metabolic and/or toxicological profiles that make them particularly suitable for in vivo administration to a warm blooded animal, such as man.

Accordingly, the present invention provides a compound of formula (I):

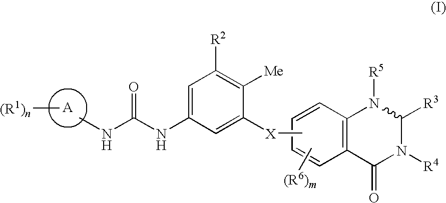

wherein:

Ring A is carbocyclyl or heterocyclyl; wherein if said heterocyclyl contains an —NH-moiety that nitrogen may be optionally substituted by a group selected from R⁷;

R¹ is a substituent on carbon and is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, N-(C₁₋₆alkoxy)sulphamoyl, N-(C₁₋₆alkyl)-N-(C₁₋₆alkoxy)sulphamoyl, C₁₋₆alkylsulphonylamino, carbocyclyl-R⁸- or heterocyclyl-R⁹-; wherein R¹ may be optionally substituted on carbon by one or more R¹⁰; and wherein if said heterocyclyl contains an —NH- moiety that nitrogen may be optionally substituted by a group selected from R¹¹;

n is selected from 0-4; wherein the values of R¹ may be the same or different;

R² is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C-₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹²- or heterocyclyl-R¹³-; wherein R² may be optionally substituted on carbon by one or more R¹⁴; and wherein if said heterocyclyl contains an —NH- moiety that nitrogen may be optionally substituted by a group selected from R¹⁵;

X is NR¹⁶ or O;

R³ and R⁶ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹⁷- or heterocyclyl-R¹⁸-; wherein R³ and R⁶ independently of each other may be optionally substituted on carbon by one or more R¹⁹; and wherein if said heterocyclyl contains an —NH- moiety that nitrogen may be optionally substituted by a group selected from R²⁰;

R⁴, R⁵ and R¹⁶ are independently selected from hydrogen, C₁₋₆alkyl, C₁₋₆alkanoyl, C₁₋₆alkylsulphonyl, C₁₋₆alkoxycarbonyl, carbamoyl, carbocyclyl, heterocyclyl, N-(C₁₋₆alkyl)carbamoyl and N,N-(C₁₋₆alkyl)carbamoyl; wherein R⁴, R⁵ and R¹⁶ independently of each other may be optionally substituted on carbon by one or more R²¹;

m is 3; wherein the values of R⁶ may be the same or different;

the bond

[symbol]

between the —NR⁵- and —CR³- of formula (I) is either (i) a single bond wherein R⁵ is as defined above, or (ii) a double bond wherein R⁵ is absent;

R¹⁰, R¹⁴, R¹⁹ and R²¹ are independently selected from halo, nitro, cyano, hydroxy, amino, carboxy, carbamoyl, mercapto, sulphamoyl, C₁₋₆aklyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C_{1- 6}alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N-(CI₁₋₆alkyl)sulphamoyl, N,N-(C₁₋₆alkyl)₂sulphamoyl, C₁₋₆alkylsulphonylamino, carbocyclyl-R²²- or heterocyclyl-R²³-; wherein R¹⁰, R¹⁴, R¹⁹ and R²¹ independently of each other may be optionally substituted on carbon by one or more R²⁴; and wherein if said heterocyclyl contains an —NH- moiety that nitrogen may be optionally substituted by a group selected from R²⁵;

R⁸, R⁹, R¹², R¹³, R¹⁷, R¹⁸, R²² and R²³ are independently selected from a direct bond, —O—, —N(R²⁶)—, —C(O)—, —N(R²⁷)C(O)—, —C(O)N(R²⁸)—, —S(O)ₛ, —SO₂N(R²⁹)- or —N(R³⁰)SO₂-; wherein R²⁶, R²⁷, R²⁸, R²⁹ and R³⁰ is hydrogen, C₁₋₆alkoxycarbonyl or C₁₋₆alkyl and s is 0-2;

R⁷, R¹¹, R¹⁵, R²⁰ and R²⁵ are independently selected from C₁₋₆alkyl, C₁₋₆alkanoyl, C₁₋₆alkylsulphonyl, C₁₋₆alkoxycarbonyl, carbamoyl, N-(C₁₋₆alkyl)carbamoyl, N,N-(C₁₋₆alkyl)carbamoyl, benzyl, benzyloxycarbonyl, benzoyl and phenylsulphonyl;

R²⁴ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N.N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

In this specification the term “alkyl” includes both straight and branched chain alkyl groups. References to individual alkyl groups such as “propyl” are specific for the straight chain version only and references to individual branched chain alkyl groups such as ‘isopropyl’ are specific for the branched chain version only. For example, “C₁₋₆alkyl” includes C₁₋₄alkyl, C₁₋₃alkyl, propyl, isopropyl and t-butyl. A similar convention applies to other radicals, for example “phenylC₁₋₆alkyl” includes phenylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a -CH₂- group can optionally be replaced by a —C(O)—, and a ring sulphur atom may be optionally oxidised to form the S-oxides. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. A particular example of the term “heterocyclyl” is pyrazolyl. In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a -CH₂- group can optionally be replaced by a —C(O)— and a ring sulphur atom may be optionally oxidised to form the S-oxides.

A “carbocyclyl” is a saturated, partially saturated or unsaturated, mono or bicyclic carbon ring that contains 3-12 atoms; wherein a -CH₂- group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or I -oxoindanyl. A particular example of “carbocyclyl” is phenyl.

An example of “C₁₋₆alkanoyloxy” is acetoxy. Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N-(C₁₋₆alkyl)amino” include methylamino and ethylamino. Examples of “N,N-(C₁₋₆alkyl)₂amino” include di-N-methylamino, di-(N-ethyl)amino and N-ethyl-N-metlhylamino. Examples of “C₂₋₆alkenyl” are vinyl, allyl and 1-propenyl. Examples of “C₂₋₆alkynyl” are ethynyl, 1-propynyl and 2-propynyl. Examples of “N-(C₁₋₆alkyl)sulphamoyl” are N-(methyl)sulphamoyl and N-(ethyl)sulphamoyl. Examples of “N-(C₁₋₆alkyl)₂sulphamoyr” are N,N-(dimethyl)sulphamoyl and N-(metliyl)-N-(ethyl)sulphamoyl. Examples of “N-(C₁₋₆alkyl)carbamoyl” are N-(C₁₋₄alkyl)carbamoyl, methylaminocarbonyl and ethylaminocarbonyl. Examples of “N,N-(C₁₋₆alkyl)₂carbamoyl” are N,N-(C₁₋₄alkyl)₂carbamoyl, dimethylaminocarbonyl and methylethylaminocarbonyl. Examples of “C₁₋₆alkylsulphonyl” are mesyl, ethylsulphonyl and isopropylsulphonyl. Examples Of “C₁₋₆alkylsulphonylamino” are mesylamino, ethylsulphonylamino and isopropylsulphonylamino. Examples of “N-(C₁₋₆alkoxy)sulphamoyl” include N-(methoxy)sulphamoyl and N-(ethoxy)sulphamoyl. Examples of “N-(C₁₋₆alkyl)-N-(C₁₋₆alkoxy)sulphamoyl” N-(methyl)-N-(methoxy)sulphamoyl and N-(propyl)-N-(ethoxy)sulphamoyl.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess B-Raf inhibitory activity. The invention further relates to any and all tautomeric forms of the compounds of the formula (I) that possess B-Raf inhibitory activity.

It is also to be understood that certain compounds of the formula (I) can exist in solvated as well as unsolvated forms such as, for example, hydrated forms. It is to be understood that the invention encompasses all such solvated forms which possess B-Raf inhibitory activity.

Particular values of variable groups are as follows. Such values may be used where appropriate with any of the definitions, claims or embodiments defined hereinbefore or hereinafter.

Ring A is carbocyclyl.

Ring A is heterocyclyl; wherein if said heterocyclyl contains an —NH- moiety that nitrogen may be optionally substituted by a group selected from R⁷.

Ring A is phenyl.

R¹ is a substituent on carbon and is selected from halo, cyano or C₁₋₆alkyl; wherein R¹ may be optionally substituted on carbon by one or more R¹⁰; wherein R¹⁰ is selected from halo or cyano.

R¹ is a substituent on carbon and is selected from fluoro, chloro, cyano, methyl or isopropyl; wherein R¹ may be optionally substituted on carbon by one or more R¹⁰; wherein R¹⁰ is selected from halo or cyano.

R¹ is a substituent on carbon and is selected from fluoro, chloro, cyano, trifluoromethyl or 1-cyano-1-methylethyl.

n is selected from 1 or 2; wherein the values of R¹ may be the same or different.

R² is hydrogen.

X is NR⁶.

X is NH.

X is O.

R³ and R⁶ are hydrogen.

R⁴ is C₁₋₆alkyl.

R⁴ is methyl.

the bond

[symbol]

between the —NR⁵- and —CR³- of formula (I) is a single bond wherein R⁵ is as defined above.

the bond

[symbol]

between the —NR⁵- and —CR³- of formula (I) is a double bond wherein R⁵ is absent.

Therefore in a further aspect of the invention there is provided a compound of formula (I) (as depicted above) wherein:

Ring A is carbocyclyl;

R¹ is a substituent on carbon and is selected from halo, cyano or C₁₋₆alkyl; wherein R¹ may be optionally substituted on carbon by one or more R¹⁰; wherein R¹⁰ is selected from halo or cyano;

n is selected from 1 or 2; wherein the values of R¹ may be the same or different;

R² is hydrogen;

X is NH;

R³ and R⁶ are hydrogen;

R⁴ is C₁₋₆alkyl;

m is 3; wherein the values of R⁶ may be the same or different;

the bond

[symbol]

between the —NR⁵- and —CR³- of formula (I) is a double bond wherein R⁵ is absent;
or a pharmaceutically acceptable salt thereof.

Therefore in a further aspect of the invention there is provided a compound of formula (I) (as depicted above) wherein:

Ring A is phenyl;

R¹ is a substituent on carbon and is selected from fluoro, chloro, cyano, trifluoromethyl or 1-cyano-1-methylethyl;

n is selected from 1 or 2; wherein the values of R¹ may be the same or different;

R² is hydrogen;

X is NH;

R³ and R⁶ are hydrogen;

R⁴ is methyl;

m is 3; wherein the values of R⁶ may be the same or different;

the bond

[symbol]

between the —NR⁵- and —CR³- of formula (I) is a double bond wherein R⁵ is absent;
or a pharmaceutically acceptable salt thereof.

In another aspect of the invention, preferred compounds of the invention are any one of the Examples or a pharmaceutically acceptable salt thereof.

Another aspect of the present invention provides a process for preparing a compound of formula (I) or a pharmaceutically acceptable salt thereof which process (wherein variable are, unless otherwise specified, as defined in formula (I)) comprises of:

Process a) reacting an amine of the formula (II):

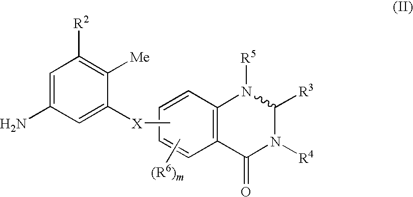

with an isocyanato of formula (III):

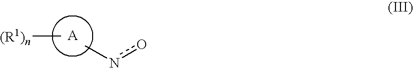

Process b) reacting a compound of fonnula (IV):

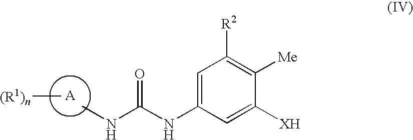

with an compound of formula (V):

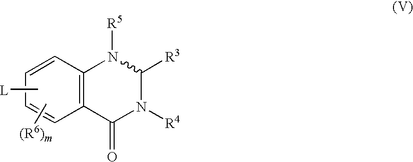

wherein L is a displaceable group;
Process c) reacting a compound of formula (VI):

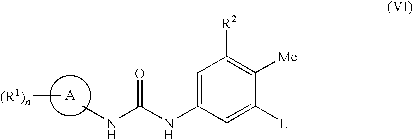

wherein L is a displaceable group; with an compound of formula (VII):

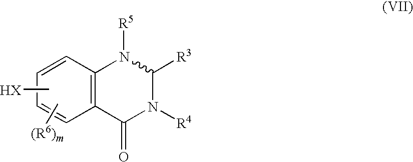

Process d) for compounds of formula (I) wherein R⁴ is not hydrogen; reacting a compound of formula (I) wherein R⁴ is hydrogen with a compound of formula (VIII):

R⁴-L (VIII)

wherein L is a displaceable group and R⁴ is not hydrogen;
Process e) for compounds of formula (I) wherein X is NR¹⁶ and R¹⁶ is -CH₂-C₂₋₆alkyl optionally substituted on carbon by one or more R²¹; reacting a compound of formula (I) wherein X is NR¹⁶ and R¹⁶ is hydrogen with a compound of formula (IX):

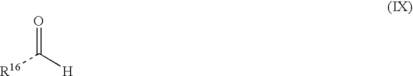

wherein R¹⁶ is C₁₋₅alkyl optionally substituted on carbon by one or more R²¹;
Process f) for compounds of formula (I) wherein X is NR¹⁶ and R¹⁶ is not hydrogen; reacting a compound of formula (I) wherein X is NR¹⁶ and R¹⁶ is hydrogen with a compound of formula (X):

R¹⁶-L (X)

wherein L is a displaceable group and R¹⁶ is not hydrogen;
Process g) reacting an isocyanato of the formula (XI):

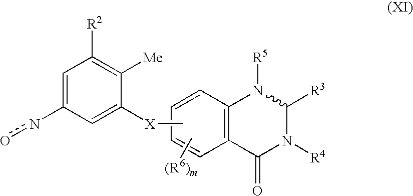

with an amine of formula (XII):

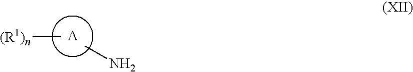

and thereafter if necessary:
i) converting a compound of the formula (I) into another compound of the formula (I);
ii) removing any protecting groups;
iii) forming a pharmaceutically acceptable salt.

L is a displaceable group, suitable values for L are for example, a halo for example a chloro or bromo.

Specific reaction conditions for the above reactions are as follows. Process a) and Process g) Isocyanatos and amines may be reacted together in an appropriate solvent such as THF or DCM from temperatures of 25 ° C. upwards.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Amines of fonnula (II) may be prepared according to Scheme 1:

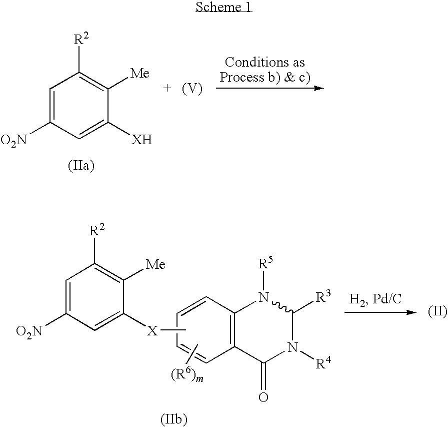

Isocyanatos of formula (XI) may be prepared by reacting a compound of formula (II) and triphosgene under standard conditions.

Compounds of formula (IIa), (III) and (XII) are commercially available compounds, or they are known in the literature or they may be prepared by standard processes known in the art.

Process b) and Process c) Compounds of formula (IV) and (V) and compounds of formula (VI) and (VII) can be reacted together by coupling chemistry utilizing an appropriate catalyst and ligand such as Pd₂(dba)₃ and BINAP respectively and a suitable base such as sodium tert-butoxide. The reaction usually requires thermal conditions often in the range of 80° C. to 100° C.

Compounds of formula (IV) may be prepared according to Scheme 2:

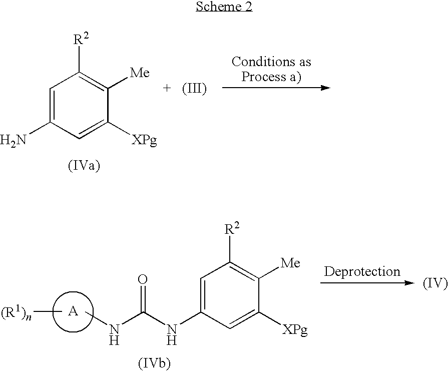

wherein Pg is a suitable protecting group.

Compounds of formula (VI) may be prepared according to Scheme 3:

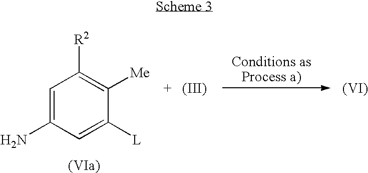

wherein Pg is a suitable protecting group.

Compounds of formula (IVa), (V), (VIa) and (VII) are commercially available compounds, or they are known in the literature or they may be prepared by standard processes known in the art.

Process d) Compounds of formula (I) and (VIII) can be reacted together in solvents such as DMF or CH₃CN in the presence of a base such as K₂CO₃ or Cs₂CO₃. The reaction usually requires thermal conditions in the range of 50° C. to 100° C.

Compounds of fonnula (VIII) are commercially available compounds, or they are known in the literature or they may be prepared by standard processes known in the art. Process e) Compounds of formula (I) and (IX) can be reacted by standard reductive amination chemistry utilizing an appropriate solvent such as THF, dichloroethane or CH₃CN, in a pH range of 6-8 using a reducing agent such as sodium triacetoxyborohydride or sodium cyanoborohydride. The reaction is typically accomplished at 25° C. This reaction can also be achieved by utilizing formic acid. The reaction usually requires thermal conditions such as 70° C.

Compounds of formula (IX) are commercially available compounds, or they are known in the literature or they may be prepared by standard processes known in the art.

Process f) Compounds of formula (I) and (X) can be reacted together in various solvents such as DMF or CH₃CN in the presence of a base such as K₂CO₃ or Cs₂CO₃. The reaction usually requires thermal conditions in the range of 50° C. to 100° C.

Compounds of formula (X) are commercially available compounds, or they are known in the literature or they may be prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T.W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses anti-cancer activity which is believed to arise from the B-Raf inhibitory activity of the compound. These properties may be assessed, for example, using the procedure set out below:-

### B-Raf in Vitro ELISA Assay

Activity of human recombinant, purified wild type His-B-Raf protein kinase was determined in vitro using an enzyme-linked immunosorbent assay (ELISA) assay format, which measures phosphorylation of the B-Raf substrate, human recombinant, purified His-derived (detagged) MEK1. The reaction utilized 2.5nM B-Raf, 0.15 μM MEK1 and 10 μM adenosine triphosphate (ATP) in 40 mM N-(2-hydroxyethyl)piperazine-N′-(2-ethanesulfonic acid hemisodium salt (HEPES), 5 mM 1,4-dithio-DL-threitol (DTT), 10 MM MgCl₂, 1 mM ethylenediaminetetraacetic acid (EDTA) and 0.2M NaCl (1× HEPES buffer), with or without compound at various concentrations, in a total reaction volume of 25μl in 384 well plates. B-Raf and compound were preincubated in 1× HEPES buffer for 1 hour at 25° C. Reactions were initiated with addition of MEK1 and ATP in 1× HEPES buffer and incubated at 25° C. for 50 minutes and reactions stopped by addition of 10 μl 175 mM EDTA (fmal concentration 50 mM) in 1× HEPES buffer. 5 μl of the assay mix was then diluted 1:20 into 50 mM EDTA in 1× HEPES buffer, transferred to 384 well black high protein binding plates and incubated for 12 h at 4° C. Plates were washed in tris buffered saline containing 0.1% Tween20 (TBST), blocked with 50 μl Superblock (Pierce) for 1 hour at 25° C., washed in TBST, incubated with 50 μl rabbit polyclonal anti-phospho-MEK antibody (Cell Signaling) diluted 1:1000 in TBS for 2 h at 25° C. , washed with TBST, incubated with 50 μl goat anti-rabbit horseradish peroxidase -linked antibody (Cell Signaling) diluted 1:2000 in TBS for 1 hour at 25° C. and washed with TBST. 50 μl of fluorogenic peroxidase substrate (Quantablu-Pierce) was added and following incubation for 45-60 mins, 50 μl QuantabluSTOP (Pierce) was added. Blue fluorescent product was detected at excitation 325 nm and emission 420 nm using a TECAN Ultra plate reader. Data was graphed and IC₅₀s calculated using Excel Fit (Microsoft).

### B-Raf In-vitro AlphaScreen Assay

Activity of purified full length His-tagged Mutant B-Raf (V600E) enzyme (MT B-Raf) was determined in-vitro using an Amplified Luminescent Proximity Homogeneous Assay (ALPHA) (Perkin Elmer, MA), which measures phosphorylation of the MT B-Raf substrate, biotinylated HIS-MEK-AVI (PLAZA internal database, construct #pAZB0141), as described below. MT B-Raf was expressed in insect cells and affinity purified by Ni⁺² agarose followed by Q-Sepharose chromatography. Typical yield was 1.08 mg/ml at >90% purity.

The phosphorylation of the MT B-Raf substrate in the presence and absence of the compound of interest was determined. Briefly, 5 μl of enzyme/substrate/adenosine triphosphate (ATP) mix consisting of 0.12 nM MT B-Raf, 84 nM biotinylated HIS-MEK-AVI, and 24 μM ATP in 1.2× buffer was preincubated with 2 μl of compound for 20 minutes at 25° C. Reactions were initiated with 5 μl of Metal mix consisting of 24 mM MgCl₂ in 1.2× buffer and incubated at 25° C. for 60 minutes and reactions were stopped by addition of 5 μl of Detection mix consisting of 20 mM HEPES, 102 mM ethylenediamine tetraacetic acid, 1.65 mg/ml BSA, 136 mM NaCl, 3.4 nM Phospho-MEK1/2 (Ser217/221) antibody (Catalog #9121, Cell Signaling Technology, Mass.), 40 μg/ml Streptavidin donor beads (Perkin Elmer, Mass., Catalog #6760002), and 40 μg/ml Protein A acceptor beads (Perkin Elmer, Mass., Catalog #6760137). Plates were incubated at 25° C. for 18 hours in the dark. Phosphorylated substrate was detected by an EnVision plate reader (Perkin Elmer, Mass.) 680 nm excitation, 520-620 nm emission. Data was graphed and IC₅₀s calculated using Excel Fit (Microsoft).

When tested in the above in vitro AlphaScreen assay, the compounds of the present invention exhibited activity less than 30 μM. For example the following results were obtained:

| Example No | IC₅₀ (μM) |
|---|---|
| 1 | 0.0143 |
| 2 | 0.363 |
| 6 | 0.099 |

According to a further aspect of the invention there is provided a pharmaceutical composition which comprises a compound of the formula (I), or a pharmaceutically acceptable salt thereof, as defined hereinbefore, in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 1-1000 mg/kg, and this normally provides a therapeutically-effective dose. Preferably a daily dose in the range of 10-100 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their B-Raf inhibitory properties. Accordingly the compounds of the present invention are expected to be useful in the treatment of diseases or medical conditions mediated alone or in part by B-Raf, i.e. the compounds may be used to produce a B-Raf inhibitory effect in a warm-blooded animal in need of such treatment.

Thus the compounds of the present invention provide a method for treating cancer characterised by inhibition of B-Raf, i.e. the compounds may be used to produce an anti-cancer effect mediated alone or in part by the inhibition of B-Raf.

Such a compound of the invention is expected to possess a wide range of anti-cancer properties as activating mutations in B-Raf have been observed in many human cancers, including but not limited to, melanoma, papillary thyroid tumours, cholangiocarcinomas, colon, ovarian and lung cancers. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, bladder, prostate, breast and pancreas. In particular such compounds of the invention are expected to slow advantageously the growth of primary and recurrent solid tumours of, for example, the skin, colon, thyroid, lungs and ovaries. More particularly such compounds of the invention, or a pharmaceutically acceptable salt thereof, are expected to inhibit the growth of those primary and recurrent solid tumours which are associated with B-Raf, especially those tumours which are significantly dependent on B-Raf for their growth and spread, including for example, certain tumours of the skin, colon, thyroid, lungs and ovaries. Particularly the compounds of the present invention are useful in the treatment of melanomas.

Thus according to this aspect of the invention there is provided a compound of the formula (I), or a pharmaceutically acceptable salt thereof, as defined hereinbefore for use as a medicament.

According to a further aspect of the invention there is provided the use of a compound of the formula (I), or a pharmaceutically acceptable salt thereof, as defined hereinbefore for the manufacture of a medicament for the production of a B-Raf inhibitory effect in a warm-blooded animal such as man.

According to this aspect of the invention there is provided the use of a compound of the formula (I), or a pharmaceutically acceptable salt thereof, as defined hereinbefore for the manufacture of a medicament for the production of an anti-cancer effect in a warm-blooded animal such as man.

According to a further feature of the invention, there is provided the use of a compound of the formula (I), or a pharmaceutically acceptable salt thereof, as defined herein before for the manufacture of a medicament for the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further aspect of the invention there is provided the use of a compound of the formula (I), or a pharmaceutically acceptable salt thereof, as defined hereinbefore in the production of a B-Raf inhibitory effect in a warm-blooded animal such as man.

According to this aspect of the invention there is provided the use of a compound of the formula (I), or a pharmaceutically acceptable salt thereof, as defined hereinbefore in the production of an anti-cancer effect in a warm-blooded animal such as man.

According to a further feature of the invention, there is provided the use of a compound of the formula (I), or a pharmaceutically acceptable salt thereof, as defined herein before in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a B-Raf inhibitory effect in a warm-blooded animal, such as man, in need of such treatment which comprises administering to said animal an effective amount of a compound of formula (I), or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof as defined herein before.

In a further aspect of the invention there is provided a phannaceutical composition which comprises a compound of the formula (I), or a pharmaceutically acceptable salt thereof, as defined herein before in association with a pharmaceutically-acceptable diluent or carrier for use in the production of a B-Raf inhibitory effect in a warm-blooded animal such as man.

In a further aspect of the invention there is provided a pharmaceutical composition which comprises a compound of the formula (I), or a phannaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.

The B-Raf inhibitory treatment defined hereinbefore may be applied as a sole therapy or may involve, in addition to the compound of the invention, conventional surgery or radiotherapy or chemotherapy. Such chemotherapy may include one or more of the following categories of anti-tumour agents :-

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafar, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbbl antibody cetuximab [C225]), farnesyl transferase inhibitors, MEK inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD 1839), N-(3 -ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;
(x) cell cycle inhibitors including for example CDK inhibitiors (eg flavopiridol) and other inhibitors of cell cycle checkpoints (eg checkpoint kinase); inhibitors of aurora kinase and other kinases involved in mitosis and cytokinesis regulation (eg mitotic kinesins); and histone deacetylase inhibitors; and
(xi) endothelin antagonists, including endothelin A antagonists, endothelin B antagonists and endothelin A and B antagonists; for example ZD4054 and ZD1611 (WO 96 40681), atrasentan and YM598.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of B-Raf in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous sodium sulphate; evaporation of solvent was carried out using a rotary evaporator under reduced pressure (600-4000 Pascals;
4.5-30 mmHg) with a bath temperature of up to 60 ° C.;
(iii) in general, the course of reactions was followed by TLC and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the fonn of delta values for major diagnostic protons, given in parts per million (ppm) relative to tetramethylsilane (TMS) as an internal standard, determined at 400 MHz using perdeuterio dimethyl sulphoxide (DMSO-d₆) as solvent unless otherwise indicated;
(vii) chemical symbols have their usual meanings; SI units and symbols are used;
(viii) solvent ratios are given in voluine:volume (v/v) terms; and
(ix) mass spectra were ran with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;
(x) where a synthesis is described as being analogous to that described in a previous example the amounts used are the millimolar ratio equivalents to those used in the previous example;
(xi) the following abbreviations have been used:

THF tetrahydrofuran;

DMF N,N-dimethylformamide;

EtOAc ethyl acetate;

Pd₂(dba)₃ tris(dibenzylideneacetone)dipalladium (0);

BINAP (+/−)-2,2′-bis(diphenylphosphino)- 1,1′-binaphthyl;

TFA trifluoroacetic acid;

DCM dichloromethane; and

DMSO dimethylsulphoxide;

(xii) “ISCO” refers to normal phase flash column chromatography using 12 g and 40 g pre-packed silica gel cartridges used according to the manufacturers instruction obtained from ISCO, Inc, 4700 superior street Lincoln, Nebr., USA.; and
(xiii) Parr Hydrogenator or Parr shaker type hydrogenators are systems for treating chemicals with hydrogen in the presence of a catalyst at pressures up to 5 atmospheres (60 psig) and temperatures to 80° C.

### Example 1

N-{4-Methyl-3-[(3-methyl-4-oxo-3,4-dihydroquinazolin-6-yl)amino]phenyl}-N-′[3-(trifluoromethyl)phenyl]urea

A solution of 1-isocyanato-3-(trifluoromethyl)benzene (100 mg, 0.53 mmol) in THF was treated with 6-[(5-amino-2-methylphenyl)amino]-3-methylquinazolin-4(3H)-one (Method 5; 150 mg, 0.53 mmol). The reaction mixture was stirred for 5 min at 25° C. The solvent was removed under reduced pressure and the residue was treated with EtOH resulting in a white precipitate. The solid was collected by vacuum filtration to yield 140 mg (56%) of the desired product. NMR: 8.94 (s, 1H), 8.71 (s, 1H), 8.13 (s, 1H), 7.95 (s, 2H), 7.55 -7.47 (m, 3H), 7.45-7.37 (m, 3H), 7.27 (d, 1H), 7.15 (d, 1H), 7.08 (d, 1H), 3.44 (s, 3H), 2.12 (s, 3H); m/z 467.

### Examples 2-5

The following compounds were prepared by the procedure of Example 1, using the indicated starting materials.

| Ex | Compound | NMR | m/z | SM |
|---|---|---|---|---|
| 2 | N-(3,4-Dichlorophenyl)- | 9.21 (s, 1H), 8.97 (s, 1H), | 469 | Method 5 and |
| | N′-{4-methyl-3-[(3- | 8.45 (s, 1H), 7.87-7.81 (m, | | 1,2-dichloro-4- |
| | methyl-4-oxo-3,4- | 1H), 7.56 (d, 1H), 7.51-7.41 | | isocyanato- |
| | dihydroquinazolin-6- | (m, 3H), 7.39 (s, 1H), 7.32- | | benzene |
| | yl)amino]phenyl}urea | 7.25 (m, 1H), 7.16 (d, 1H), | | |
| | | 7.06 (d, 1H), 3.47 (s, 3H), | | |
| | | 2.13 (s, 3H) | | |
| 3 | N-(3-Cyanophenyl)-N′- | 8.93 (s, 1H), 8.76 (s, 1H), | 425 | Method 5 and 3- |
| | {4-methyl-3-[(3-methyl- | 8.13 (s, 1H), 7.94 (s, 1H), | | isocyanato- |
| | 4-oxo-3,4- | 7.92 (m, 1H), 7.63 (m, 1H), | | benzonitrile |
| | dihydroquinazolin-6- | 7.53 (d, 1H), 7.40 (m, 4H), | | |
| | yl)amino]phenyl}urea | 7.15 (d, 1H), 7.07 (dd, 1H), | | |
| | | 3.44 (s, 3H), 2.13 (s, 3H) | | |
| 4 | N-[4-Fluoro-3- | 8.94 (s, 1H), 8.72 (s, 1H), | 486 | Method 5 and 1- |
| | (trifluoromethyl)phenyl]- | 8.13 (s, 1H), 7.94 (m, 2H), | | fluoro-4- |
| | N′-{4-methyl-3-[(3- | 7.59 (m, 1H), 7.52 (d, 1H), | | isocyanato-2- |
| | methyl-4-oxo-3,4- | 7.40 (m, 3H), 7.15 (d, 1H), | | (trifluoro- |
| | dihydroquinazolin-6- | 7.08 (dd, 1H), 3.44 (s, 3H), | | methyl)benzene |
| | yl)amino]phenyl}urea | 2.13 (s, 3H) | | |
| 5 | N-{4-Methyl-3-[(3- | 9.00 (s, 1H), 8.71 (s, 1H), | 468 | Method 5 and 1- |
| | methyl-4-oxo-3,4- | 8.14 (s, 1H), 7.94 (s, 1H), | | isocyanato-4- |
| | dihydroquinazolin-6- | 7.60 (m, 4H), 7.53 (d, 1H), | | (trifluoro- |
| | yl)amino]phenyl}-N′-[4- | 7.38-7.46 (m, 3H), 7.12- | | methyl)benzene |
| | (trifluoromethyl)- | 7.20 (m, 1H), 7.07 (dd, 1H), | | |
| | phenyl]urea | 3.44 (s, 3H), 2.13 (s, 3H) | | |

### Example 6

N-[3-(1-Cyano-1-methylethyl)phenyl]-N′-{4-methyl-3-[(3-methyl-4-oxo-3,4-dihydroquinazolin-6-yl)amino]phenyl}urea

A mixture of 2-(3-aminophenyl)-2-methylpropanenitrile (Method 10, 30 mg, 0.15 mmol), triethylamine (0.89 ml, 6.39 mmol) and triphosgene (51 mg, 0.17 mmol) in CHCl₃ was stirred at reflux for 3 min. 6-[(5-Amino-2-methylphenyl)amino]-3-methylquinazolin-4(3H1)-one (Method 5; 44 mg, 0.15 mmol) was then added. The reaction mixture was then stirred at reflux for 20 min. The reaction was cooled to 25° C. and then filtered. The resulting-white solid was washed with MeOH and dried under vacuum to yield 35 mg (48%) of the desired product. NMR: 8.79 (s, 1H), 8.64 (s, 1H), 8.14 (s, 1H), 7.98 (s, 1H), 7.62 (s, 1H), 7.50 (d, 1H), 7.44-7.25 (m, 5H), 7.23-7.00 (m, 3H), 3.45 (s, 3H), 2.13 (s, 3H), 1.66 (s, 6H); m/z 467.

### Preparation of Starting Materials

### Method 1

tert-Butyl (4-methyl-3-nitrophenyl)carbamate

A solution of 4-methyl-3-nitroaniline (10.0 g, 0.066 mol) was dissolved in THF (25 ml) at 65° C. Di-tert-butyl dicarbonate (17.2 g, 0.079 mol, 1.2 equiv) in THF (20 ml) was added dropwise over 30 min. The mixture was then refluxed under nitrogen for 12 h. The reaction was cooled to 25° C. and the solvent was removed under reduced pressure to give a brown oil. The oil was dissolved in hexane-EtOAc (4:1), (200 ml) and 30 g of silica gel was added to the solution. The solution was stirred for 5 min and the silica was removed by filtration. The silica was then repeatedly washed with hexane-EtOAc (4:1) until no further product was detected. The solvents were combined and concentrated under reduced pressure. The resulting yellow solid was washed with hexane and air dried to give 14.2 g of the desired product (85%). NMR (300 MHz): 8.07 (s, 1H), 7.53 (d, 1H), 7.26-7.30 (m, 1H), 6.66 (s, 1H), 2.55 (s, 3H), 1.55 (s, 9H).

### Method 2

tert-Butyl (3-amino-4-methylphenyl)carbamate

A solution of tert-butyl (4-methyl-3-nitrophenyl)carbamate (Method 1; 10.0 g, 39.6 mmol) was dissolved in EtOH (220 ml). The solution was treated with 10% Pd/C (650 mg) and placed on a Parr hydrogenator at 50 psi of hydrogen for 12 h. The resulting solution was filtered through diatomaceous earth and the solvent was removed under reduced pressure to give 8.68 g (98%). NMR (300 MHz): 6.86-6.98 (m, 2H), 6.48 (d, 1H), 6.36 (s, 1H), 3.59 (s, 2H), 2.09 (s, 3H), 1.42-1.50 (m, 9H).

### Method 3

6-Bromo-3-methylquinazolin-4(3H)-one

2-Amino-5-bromobenzoic acid (5.00 g, 0.023 mol) was reacted with N-methylformamide (40 ml) at 180° C. for 12 h. The reaction was quenched with H₂O and the resulting precipitate was collected by vacuum filtration to give 5.26 g (95%) of a yellow-white solid; m/z 240.

### Method 4

tert-Butvl {4-methyl-3-[(3-methyl-4-oxo-3,4-dihydroquinazolin-6-yl)amino]phenyl}carbamate

A stirred mixture of tert-butyl (3-amino-4-methylphenyl)carbamate (Method 2; 3.08 g, 0.0135 mmol), 6-bromo-3-methylquinazolin-4(3H)-one (Method 3; 3.24 g, 0.0135 mmol), Cs₂CO₃ (13.20 g, 0.0405 mol, 3.0 equiv), BINAP (841 mg, 1.35 mmol, 5 mol %) in dioxane (50 ml) was treated with Pd₂(dba)₃ (618 mg, 0.675 mmol). The reaction mixture was heated to 80° C. for 12 h. The reaction was then quenched with 10% NaOH(aq) and extracted with EtOAc. The organics were dried with NaCl(sat) and then Na₂SO₄(s). The organics were removed under reduced pressure and the resulting solid was treated with DCM (100 ml). The resulting precipitate was collected by vacuum filtration (3.00 g, 58%); m/z 387.

### Method 5

6-[(5-Amino-2-methylphenyl)amino]-3-methylquinazolin-4(3H)-one

A stirred mixture of tert-butyl {4-methyl-3-[(3-methyl-4-oxo-3,4-dihydroquinazolin-6-yl)amino]phenyl}carbamate (Method 4; 3.00 g, 7.78 mmol) in DCM (30 ml) was treated with TFA (30 ml). The solvents were removed under reduced pressure. The resulting solid was treated with 10% NaOH(aq) and extracted with EtOAc. The organics were dried with NaCl(sat) and then Na₂SO₄(s). The organics were then removed under reduced pressure to provide the desired product (2.18 g, 99%); m/z 280.

### Method 6

3-Cyanomethyl-benzoic acid methyl ester

A suspension of methyl-3-(bromomethyl)benzoate (13.5 g, 58.9 mmol) and sodium cyanide (4.33 g, 88.4 mmol) in DMF (25 ml) and water (1 ml) was stirred at 75° C. for 5 h. The reaction mixture was quenched with water and extracted with EtOAc. The combined organics were dried with NaCl(sat) and then Na₂SO₄(s). The solvents were removed under reduced pressure. The resulting residue was purified by column chromatography utilizing an ISCO system (hexane-EtOAc) to give 7.2 g (70%) of colourless oil. NMR: 7.90 (s, 1H), 7.86 (d, 1H), 7.60 (d, 1H), 7.50 (m, 1H), 4.10 (s, 2H), 3.80 (s, 3H); m/z 175.

### Method 7

3-(1 -Cyano-1-methylethyl)benzoic acid methyl ester

A solution of 3-cyanomethyl-benzoic acid methyl ester (Method 6; 7.2 g, 41.1 mmol) in anhydrous DMSO (80 ml) was treated with sodium hydride (60%, 4.9 g, 123.3 mmol, 3 eq). Methyl iodide was then added dropwise at 0° C. The reaction mixture was stirred at 25° C. for 12 h. The reaction mixture was then quenched with water and extracted with EtOAc. The combined organics were dried with NaCl(sat) and then Na₂SO₄(s). The solvents were removed under reduced pressure. The crude product was purified by column chromatography utilizing an ISCO system (hexane-EtOAc) to give 5.5 g (66%) of a colourless oil. NMR: 8.05 (s, 1H), 7.90 (d, 1H), 7.75 (d, 1H), 7.55 (m, 1H), 3.80 (s, 3H), 1.62 (s, 6H); m/z 203.

### Method 8

3-(1-Cyano-1-methylethyl)benzoic acid

A solution of 3-(1-cyano-1-methylethyl)benzoic acid methyl ester (Method 7; 5.5 g, 27.1 mmol) in THF/MeOW/H₂O (3:1:1, 100 ml) was treated with lithium hydroxide (1.95 g) in water (20 ml). The mixture was stirred at 25° C. for 12 h. The reaction mixture was concentrated under reduced pressure and the resulting solution was diluted with water, and then acidified with 10% HC1. The resulting white solid (4.83 g, 94%) was collected by vacuum filtration. NMR: 13.00 (s, 1H), 7.95 (s, 1H), 7.80 (d, 1H), 7.65 (d, 1H), 7.45 (m, 1H), 1.60 (s, 6H); m/z 189.

### Method 9

tertButyl[3-(1-cyano-1-methylethyl)phenyl]carbamate

A suspension of 3-(1-cyano-1-methylethyl)benzoic acid (Method 8; 189 mg, 1 mmol), diphenyl phosphoryl azide (550 mg, 2 mmol) and diisopropylethyl amine (258 mg, 2 mmol) in tert-butanol (10 ml) was refluxed for 12 h. The solvent was removed under reduced pressure. The crude product was purified by column chromatography utilizing an ISCO system (hexane-EtOAc) giving the desired product. NMR: 7.05-7.50 (m, 4H), 6.55 (s, 1H), 1.60 (s, 6H), 1.40 (s, 9H).

### Method 10

2-(3-Aminophenyl)-2-methylpropanenitrile

tert-Butyl[3-(1-cyano-1-methylethyl)phenyl]carbamate (Method 9) was treated with 4M HCl in dioxane (5 ml) and the reaction was stirred for 12 h. The resulting solid was dissolved in 10% NaOH(aq) and extracted with EtOAc. The combined organics were dried with NaCl(sat) and then Na₂SO₄(s). The solvents were removed under reduced pressure. The crude product was purified by column chromatography utilizing an ISCO system (hexane-EtOAc) providing the desired product. NMR: 6.90-7.30 (m, 4H), 1.65 (s, 6H); m/z 160.

1. A compound of formula (I): 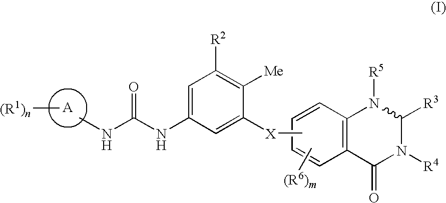
wherein:
Ring A is carbocyclyl or heterocyclyl; wherein if said heterocyclyl contains an —NH- moiety that nitrogen may be optionally substituted by a group selected from R⁷;
R¹ is a substituent on carbon and is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl,
N,N-(C₁₋₆alkyl)₂sulphamoyl, N-(C₁₋₆alkoxy)sulphamoyl,
N-(C₁₋₆alkyl)-N-(C₁₋₆alkoxy)sulphamoyl, C₁₋₆alkylsulphonylamino, carbocyclyl-R⁸- or heterocyclyl-R⁹-; wherein R¹ may be optionally substituted on carbon by one or more R¹⁰; and wherein if said heterocyclyl contains an —NH- moiety that nitrogen may be optionally substituted by a group selected from R¹¹;
n is selected from 0-4; wherein the values of R¹ may be the same or different;
R² is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ
wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹²- or heterocyclyl-R³-; wherein R² may be optionally substituted on carbon by one or more R¹⁴; and wherein if said heterocyclyl contains an —NH- moiety that nitrogen may be optionally substituted by a group selected from R¹⁵;
X is NR¹⁶or O;
R³ and R⁶ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹⁷- or heterocyclyl-R¹⁸-; wherein R³ and R⁶ independently of each other may be optionally substituted on carbon by one or more R¹⁹; and wherein if said heterocyclyl contains an —NH- moiety that nitrogen may be optionally substituted by a group selected from R²⁰;
R⁴, R⁵ and R¹⁶ are independently selected from hydrogen, C₁₋₆alkyl, C₁₋₆alkanoyl, C₁₋₆alkylsulphonyl, C₁₋₆alkoxycarbonyl, carbamoyl, carbocyclyl, heterocyclyl, N-(C₁₋₆alkyl)carbamoyl and N,N-(C₁₋₆alkyl)carbamoyl; wherein R⁴, R⁵ and R¹⁶ independently of each other may be optionally substituted on carbon by one or more R²¹;
m is 3; wherein the values of R⁶ may be the same or different;
the bond [symbol]
between the —NR⁵- and —CR³- of formula (I) is either (i) a single bond wherein R⁵ is as defined above, or (ii) a double bond wherein R⁵ is absent;
R¹⁰, R¹⁴, R¹⁹ and R²¹ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, carbocyclyl-R²²- or heterocyclyl-R²³-; wherein R¹⁰, R¹⁴, R¹⁹ and R²¹ independently of each other may be optionally substituted on carbon by one or more R²⁴; and wherein if said heterocyclyl contains an —NH- moiety that nitrogen may be optionally substituted by a group selected from R²⁵;
R⁸, R⁹, R¹², R¹³, R¹⁷, R¹⁸, R²² and R²³ are independently selected from a direct bond, —O—, —N(R²⁶)—, —C(O)—, —N(R²⁷)C(O)—, —C(O)N(R²)—, —S(O)ₛ—, —SO₂N(R²⁹)- or —N(R³⁰)SO₂—; wherein R²⁶, R²⁷, R²⁸, R²⁹ and R³⁰ is hydrogen, C₁₋₆alkoxycarbonyl or C₁₋₆alkyl and s is 0-2;
R⁷, R¹¹, R¹⁵, R²⁰ and R²⁵ are independently selected from C₁₋₆alkyl, C₁₋₆alkanoyl, C₁₋₆alkylsulphonyl, C₁₋₆alkoxycarbonyl, carbamoyl, N-(C₁₋₆alkyl)carbamoyl, N,N-(C₁₋₆alkyl)carbamoyl, benzyl, benzyloxycarbonyl, benzoyl and phenylsulphonyl;
R²⁴ is selected from halo, nitro, cyano, hydroxy, trifluoromethoxy,
trifluoromethyl, amino, carboxy, carbamoyl, mercapto, sulphamoyl, methyl, ethyl,
methoxy, ethoxy, acetyl, acetoxy, methylamino, ethylamino, dimethylamino,
diethylamino, N-methyl-N-ethylamino, acetylamino, N-methylcarbamoyl,
N-ethylcarbamoyl, N,N-dimethylcarbamoyl, N,N-diethylcarbamoyl,
N-methyl-N-ethylcarbamoyl, methylthio, ethylthio, methylsulphinyl, ethylsulphinyl,
mesyl, ethylsulphonyl, methoxycarbonyl, ethoxycarbonyl, N-methylsulphamoyl,
N-ethylsulphamoyl, N,N-dimethylsulphamoyl, N,N-diethylsulphamoyl or
N-methyl-N-ethylsulphamoyl;
or a pharmaceutically acceptable salt thereof.

2. A compound of formula (I), or a pharmaceutically acceptable salt thereof, as claimed in claim 1 wherein Ring A is phenyl.

3. A compound of formula (I), or a pharmaceutically acceptable salt thereof, as claimed in claim 1 wherein R¹ is a substituent on carbon and is selected from halo, cyano or C₁₋₆alkyl; wherein R¹ may be optionally substituted on carbon by one or more R¹⁰; wherein R¹⁰ is selected from halo or cyano.

4. A compound of formula (I), or a pharmaceutically acceptable salt thereof, as claimed in claim 1 wherein n is selected from 1 or 2; wherein the values of R¹ may be the same or different.

5. A compound of formula (I), or a pharmaceutically acceptable salt thereof, as claimed in claim 1 wherein R² is hydrogen.

6. A compound of formula (I), or a pharmaceutically acceptable salt thereof, as claimed in claim 1 wherein X is NH.

8. A compound of formula (I), or a pharmaceutically acceptable salt thereof, as claimed in claim 16 wherein R³ and R⁶ are hydrogen.

9. A compound of formula (I), or a pharmaceutically acceptable salt thereof, as claimed in claim 1 wherein R⁴ is C₁₋₆alkyl.

10. A compound of formula (I), or a pharmaceutically acceptable salt thereof, as claimed in claims 1 wherein the bond [symbol]
between the —NR⁵- and —CR³- of formula (I) is a double bond wherein R⁵ is absent.

11. A compound of formula (I): 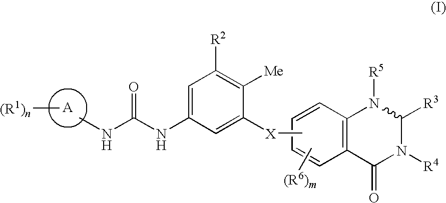
wherein:
Ring A is phenyl;
R¹ is a substituent on carbon and is selected from fluoro, chloro, cyano, trifluoromethyl or 1-cyano-1-methylethyl;
n is selected from 1 or 2; wherein the values of R¹ may be the same or different;
R² is hydrogen;
X is NH;
R³ and R⁶ are hydrogen;
R⁴ is methyl;
m is 3; wherein the values of R⁶ may be the same or different;
the bond [symbol]
between the —NR⁵- and —CR³- of formula (I) is a double bond wherein R⁵is absent;
or a pharmaceutically acceptable salt thereof.

12. A compound of formula (I): 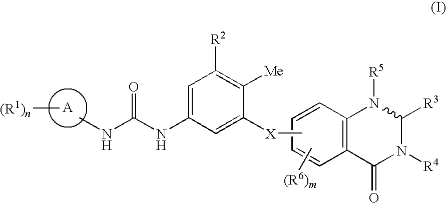
selected from:
N-{4-methyl-3-[(3-methyl-4-oxo-3,4-dihydroquinazolin-6-yl)amino]phenyl }-N′-[3-(trifluoromethyl)phenyl]urea;
N-(3,4-dichlorophenyl)-N′- {4-methyl-3-[(3 -methyl-4-oxo-3,4-dihydroquinazolin-6-yl)amino]phenyl}urea;
N-(3-cyanophenyl)-N′- {4-methyl-3-[(3-methyl-4-oxo-3,4-dihydroquinazolin-6-yl)amino]phenyl}urea;
N-[4-fluoro-3-(trifluoromethyl)phenyl]-N′-{4-methyl-3-[(3-methyl-4-oxo-3,4-dihydroquinazolin-6-yl)amino]phenyl}urea;
N-{4-methyl-3-[(3-methyl-4-oxo-3,4-dihydroquinazolin-6-yl)amino]phenyl}-N′-[4-(trifluoromethyl)-phenyl]urea; and
N-[3-(1-cyano-1-methylethyl)phenyl]-N′-{4-methyl-3-[(3-methyl-4-oxo-3,4-dihydroquinazolin-6-yl)amino]phenyl}urea;
or a pharmaceutically acceptable salt thereof.

13. (canceled)

14. A pharmaceutical composition which comprises a compound of the formula (I), or a pharmaceutically acceptable salt thereof, as claimed in claim 1, in association with a pharmaceutically-acceptable diluent or carrier.

15-19. (canceled)

20. A method for producing an anti-cancer effect in a warm-blooded animal, such as man, in need of such treatment which comprises administering to said animal an effective amount of a compound of formula (I), or a pharmaceutically acceptable salt thereof, as claimed in claim 1.

21. A method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof, as claimed in claim 1.

22-24. (canceled)
